(19) United States
(12) Patent Application Publication
(10) Pub. No.: US 2024/0148819 A1
(43) Pub. Date: May 9, 2024

(54) COMPOUNDS FOR USE IN THE TREATMENT OF SINUSITIS, PNEUMONIA OR OTITIS

(71) Applicant: PHARMA HOLDINGS AS, Tromsø (NO)

(72) Inventors: Christian LÜTKEN, Oslo (NO); Johnny Ivar RYVOLL, Tromsø (NO); Torsteinn ERLINGSSON, Tromsø (NO)

(73) Assignee: PHARMA HOLDINGS AS, Tromsø (NO)

(21) Appl. No.: 18/546,799

(22) PCT Filed: Mar. 18, 2022
(86) PCT No.: PCT/EP2022/057213

§ 371 Date: Aug. 17, 2023

(30) Foreign Application Priority Data
Mar. 26, 2021 (GB) 2104348.4
Oct. 25, 2021 (GB) 2115327.5

(51) Int. Cl. A61K 38/06; A61P 31/04; A61P 31/10; A61P 31/16

(52) U.S. Cl. CPC A61K 38/06; A61P 31/04; A61P 31/10; A61P 31/16

(57) ABSTRACT

The present invention provides a compound for use in the treatment of sinusitis, pneumonia or otitis in a subject, wherein said compound is a compound of Formula (I) as defined herein: AA-AA-AA-X—Y—Z (I). The invention further provides a method of treating sinusitis, pneumonia or otitis in a subject, which method comprises administering to a subject in need thereof an effective amount of a compound of Formula (I).

The present invention relates to the treatment of certain diseases/conditions. More particularly, the invention relates to the use of certain compounds for the treatment of sinusitis, pneumonia or otitis.

Many conditions (or diseases) are caused by pathogens, in particular pathogenic microorganisms such as bacteria and viruses. Some conditions (or diseases) caused by pathogens are caused by only one particular pathogen, or are caused by only one particular type of pathogen. For example, some diseases are caused only by bacteria, and are not caused by viruses. Conversely, some other diseases are caused only by viruses, and are not caused by bacteria.

However, some diseases can be caused by multiple different types of causative agent. For example, the causative agent (or infectious agent) of sinusitis, pneumonia and otitis media can be a bacteria or a virus. Thus, in some cases, sinusitis, pneumonia and otitis media are caused by bacteria, but in other cases sinusitis, pneumonia and otitis media are caused by viruses. In some other cases, both bacteria and viruses may be present in cases of sinusitis, pneumonia and otitis media, in so-called polymicrobial infections. In a small proportion of cases of sinusitis, pneumonia and otitis media, the causative agent of the disease may be a fungus.

Antibacterial agents and antiviral agents are known, but these two classes of agents, targeting these two different types of pathogenic microorganisms, are typically very different from one another. Furthermore, antibacterial agents are not typically active against viruses, and vice versa. In this regard, it is well-known that antibiotics are not effective agents for the treatment of viral infections. Likewise, antiviral agents (e.g. entry inhibitors, uncoating inhibitors, release (or exit) inhibitors, etc.) are not effective agents for the treatment of bacterial infections.

In patients having a condition that may be caused by multiple different types of causative agents, such as sinusitis, pneumonia and otitis, it can be very difficult to ascertain the relevant causative agent in a given case (e.g. when presenting to a clinician with the condition), for example because the symptoms can be substantially the same irrespective of the causative agent. Laboratory tests can be performed to try to identify the causative agent, but this is time consuming and costly. In the case of sinusitis, pneumonia and otitis, upon presentation of a patient it can be determined that a patient has the relevant disease but it is difficult to definitively determine the underlying causative agent of the disease. Without knowing the underlying causative agent, clinicians are unable to make fully informed decisions as to appropriate treatment strategies to employ.

It is clear that if compounds could be identified as having antibacterial activity and antiviral activity and antifungal activity against viruses and bacteria and fungi that are clinically relevant to sinusitis, pneumonia and otitis, that would be highly desirable and advantageous. In that case, sinusitis, pneumonia and otitis could be treated with such compounds without needing to know the underlying causative agent of the disease. Such compounds would be effective in the treatment of sinusitis, pneumonia and otitis irrespective of whether the cause of the disease was a bacterial infection, a viral infection, or a fungal infection (or a polymicrobial infection in which more than one type of causative agent is present, e.g. bacteria and virus).

The present inventors have surprisingly found that a class of tripeptide compounds that carry a certain C-terminal modification exhibit excellent antiviral activity against viruses that are causative agents of sinusitis, pneumonia and otitis, in addition to exhibiting excellent activity against bacteria and fungi that are causative agents of sinusitis, pneumonia and otitis. Such tripeptides are cationic (positively charged) and bulky. One compound in this class is the compound LTX-109.

Given the findings of the present inventors, such compounds clearly represent an important class of agent to be added to the current arsenal of therapies for sinusitis, pneumonia and otitis.

Thus, in one aspect, the present invention provides a compound for use in the treatment of sinusitis, pneumonia or otitis in a subject, wherein said compound is a compound of Formula (I)

AA-AA-AA-X—Y—Z (I)
wherein, in any order, 2 of said AA (amino acid) moieties are cationic amino acids, preferably lysine or arginine but may be histidine or any non-genetically coded or modified amino acid carrying a positive charge at pH 7.0, and 1 of said AA is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms (in the case of fused rings of course the non-hydrogen atoms may be shared);
X is a N atom, which may be, but preferably is not, substituted by a branched or unbranched C₁-C₁₀ alkyl or aryl group, e.g. methyl, ethyl or phenyl, and this group may incorporate up to 2 heteroatoms selected from N, O and S;
Y represents a group selected from —Rₐ—R_{b}—, —Rₐ—R_{b}—R_{b}— and —R_{b}—R_{b}—Rₐ— wherein
Rₐ is C, O, S or N, preferably C, and
R_{b} is C; each of Rₐ and R_{b} may be substituted by C₁-C₄ alkyl groups or unsubstituted, preferably Y is —Rₐ—R_{b}— (in which Rₐ is preferably C) and preferably this group is not substituted, when Y is —Rₐ—R_{b}—R_{b}— or —R_{b}—R_{b}—Rₐ— then preferably one or more of Rₐ and R_{b} is substituted; and
Z is a group comprising 1 to 3 cyclic groups each of 5 or 6 non-hydrogen atoms (preferably C atoms), 2 or more of the cyclic groups may be fused; one or more of the rings may be substituted and these substitutions may, but will typically not, include polar groups, suitable substituting groups include halogens, preferably bromine or fluorine and C₁-C₄ alkyl groups; the Z moiety incorporates a maximum of 15 non-hydrogen atoms, preferably 5-12, most preferably it is phenyl;
the bond between Y and Z is a covalent bond between Rₐ or R_{b} of Y and a non-hydrogen atom of one of the cyclic groups of Z.

Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethyllysine and trimethylornithine, 4-aminopiperidine-4-carboxylic acid, 4-amino-1-carbamimidoylpiperidine-4-carboxylic acid and 4-guanidinophenylalanine.

The large lipophilic R group of the AA may contain hetero atoms such as O, N or S, typically there is no more than one heteroatom, preferably it is nitrogen. This R group will preferably have no more than 2 polar groups, more preferably none or one, most preferably none.

Compounds for use in accordance with the invention are preferably peptides.

Compounds for use in accordance with the invention are preferably of formula (II)

AA₁-AA₂-AA₁-X—Y—Z (II)
wherein:
AA₁ is a cationic amino acid, preferably lysine or arginine but may be histidine or any non-genetically coded or modified amino acid carrying a positive charge at pH 7.0;
AA₂ is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms; and
X, Y and Z are as defined above.

Further preferred compounds for use in accordance with the invention include compounds of formulae (III) and (IV):

AA₂-AA₁-AA₁-X—Y—Z (III)

AA₁-AA₁-AA₂-X—Y—Z (IV)
wherein AA₁, AA₂, X, Y and Z are as defined above. Molecules of formula (II) are more preferred.

From amongst the above compounds certain are particularly preferred. In particular, compounds wherein the amino acid with a large lipophilic R group, conveniently referred to herein as AA₂, is tributyl tryptophan (Tbt) or a biphenylalanine derivative such as Phe(4-(2-Naphthyl)), Phe(4-(1-Naphthyl)), Bip (4-n-Bu), Bip (4-Ph) or Bip (4-T-Bu); Phe(4-(2-Naphthyl)), Phe(4-(1-Naphthyl)) and Tbt being most preferred. In some preferred embodiments, the amino acid with a lipophilic R group is tributyl tryptophan (Tbt).

In some preferred embodiments, Y is —Rₐ—R_{b}— and unsubstituted, most preferably Rₐ and R_{b} are both carbon (C) atoms. Preferably, Y is —CH₂—CH₂—.

In some preferred embodiments, Z is phenyl (Ph).

A further preferred group of compounds are those in which —X—Y—Z together is the group —NHCH₂CH₂Ph.

The compounds include all enantiomeric forms, both D and L amino acids and enantiomers resulting from chiral centers within the amino acid R groups and the C-terminal capping group “—X—Y—Z”. β and γ amino acids as well as α amino acids are included within the term ‘amino acids’, as are N-substituted glycines which may all be considered AA units. The compounds for use in accordance with the invention include beta peptides and depsipeptides.

The most preferred compound has the structural formula:

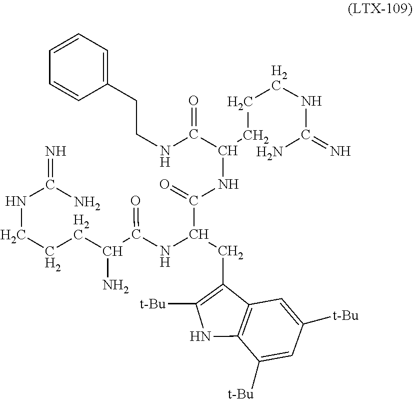 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

t-Bu represents a tertiary butyl group. This compound with the structural formula above incorporating the amino acid 2,5,7-Tris-tert-butyl-L-tryptophan is the most preferred compound for use in the present invention (and is also referred to herein as LTX-109). Analogues of this compound incorporating other cationic residues in place of Arg, in particular Lys, are also highly preferred. Analogues incorporating alternative C terminal capping groups as defined above are also highly preferred.

Another preferred compound for use in accordance with the present invention is:

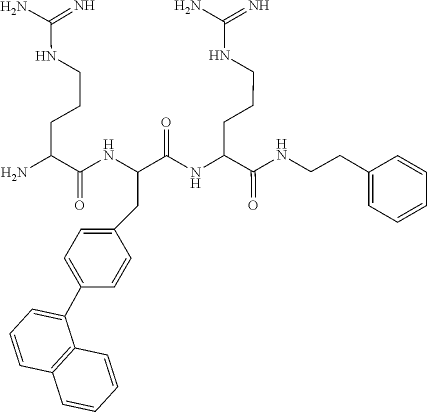 [SMILES: N=C(N)NCCCC(N)C(=O)NC(Cc1ccc(-c2cccc3ccccc23)cc1)C(=O)NC(CCCNC(=N)N)C(=O)NCCc1ccccc1]

This compound (i.e. the compound with the structural formula depicted immediately above) may be referred to as Arg-Phe(4-(1-Naphthyl))-Arg-NH—CH₂—CH₂-Ph. This compound is a compound of formula (II) in which AA, is arginine (Arg), AA₂ is Phe(4-(1-Naphthyl)), and —X—Y—Z together is the group —NHCH₂CH₂Ph.

Another preferred compound for use in accordance with the present invention is:

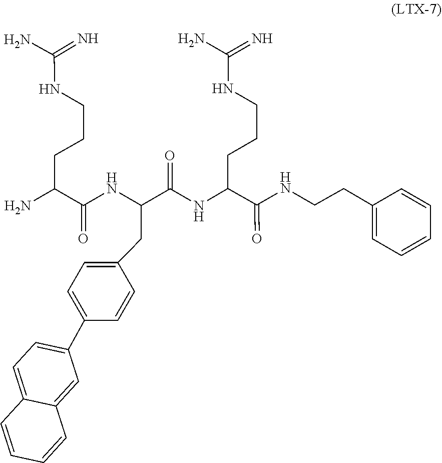 [SMILES: N=C(N)NCCCC(N)C(=O)NC(Cc1ccc(-c2ccc3ccccc3c2)cc1)C(=O)NC(CCCNC(=N)N)C(=O)NCCc1ccccc1]

This compound (i.e. the compound with the structural formula depicted immediately above) may be referred to as Arg-Phe(4-(2-Naphthyl))-Arg-NH—CH₂—CH₂-Ph. This compound is also referred to herein as LTX-7. This compound is a compound of formula (II) in which AA, is arginine (Arg), AA₂ is Phe(4-(2-Naphthyl)), and —X—Y—Z together is the group —NHCH₂CH₂Ph.

Another preferred compound for use in accordance with the present invention is:

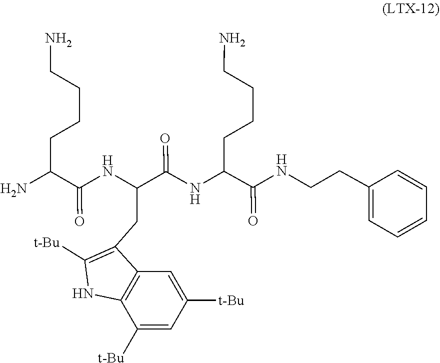 [SMILES: CC(C)(C)c1cc(C(C)(C)C)c2[nH]c(C(C)(C)C)c(CC(NC(=O)C(N)CCCCN)C(=O)NC(CCCCN)C(=O)NCCc3ccccc3)c2c1]

t-Bu represents a tertiary butyl group. This compound (i.e. the compound with the structural formula depicted immediately above) may be referred to as Lys-Tbt-Lys-NH—CH₂—CH₂-Ph. This compound may also be referred to as LTX-12. This compound is a compound of formula (II) in which AA, is lysine (Lys), AA₂ is tributyl tryptophan (Tbt, which may also be referred to as 2,5,7-Tris-tert-butyl-L-tryptophan), and —X—Y—Z together is the group —NHCH₂CH₂Ph.

In some embodiments, the compound for use in accordance with the present invention is selected from the group consisting of LTX-109, LTX-7 and LTX-12. The compound LTX-109 is the most preferred compound for use in accordance with the present invention.

Compounds for use in the present invention are preferably peptides.

The compounds of formulae (I) to (IV) may be peptidomimetics and peptidomimetics of the peptides described and defined herein also represent compounds of use in accordance with the present invention. A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. Suitable amide bond surrogates include the following groups: N-alkylation (Schmidt, R. et al., Int. J. Peptide Protein Res., 1995, 46,47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

The peptidomimetic compounds of use in the present invention will typically have 3 identifiable sub-units which are approximately equivalent in size and function to amino acids (AA units). The term ‘amino acid’ may thus conveniently be used herein to refer to the equivalent sub-unit of a peptidomimetic compound. Moreover, peptidomimetics may have groups equivalent to the R groups of amino acids and discussion herein of suitable R groups and of N and C terminal modifying groups applies, mutatis mutandis, to peptidomimetic compounds.

As is discussed in the text book referenced above, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142. Strongly basic conditions will favour N-methylation over O-methylation and result in methylation of some or all of the nitrogen atoms in the peptide bonds and the N-terminal nitrogen.

Preferred peptidomimetic backbones include polyesters, polyamines and derivatives thereof as well as substituted alkanes and alkenes. The peptidomimetics will preferably have N and C termini which may be modified as discussed herein.

The compounds for use in the invention may be synthesised in any convenient way. Generally the reactive groups present (for example amino, thiol and/or carboxyl) will be protected during overall synthesis. The final step in the synthesis will thus be the deprotection of a protected derivative of the invention.

In building up a peptide, one can in principle start either at the C-terminal or the N-terminal although the C-terminal starting procedure is preferred.

Methods of peptide synthesis are well known in the art but for the present invention it may be particularly convenient to carry out the synthesis on a solid phase support, such supports being well known in the art.

A wide choice of protecting groups for amino acids are known and suitable amine protecting groups may include carbobenzoxy (also designated Z) t-butoxycarbonyl (also designated Boc), 4-methoxy-2,3,6-trimethylbenzene sulphonyl (Mtr) and 9-fluorenylmethoxy-carbonyl (also designated Fmoc). It will be appreciated that when the peptide is built up from the C-terminal end, an amine-protecting group will be present on the α-amino group of each new residue added and will need to be removed selectively prior to the next coupling step.

Carboxyl protecting groups which may, for example be employed include readily cleaved ester groups such as benzyl (Bzl), p-nitrobenzyl (ONb), pentachlorophenyl (OPCIP), pentafluorophenyl (OPfp) or t-butyl (OtBu) groups as well as the coupling groups on solid supports, for example methyl groups linked to polystyrene.

Thiol protecting groups include p-methoxybenzyl (Mob), trityl (Trt) and acetamidomethyl (Acm).

A wide range of procedures exists for removing amine- and carboxyl-protecting groups. These must, however, be consistent with the synthetic strategy employed. The side chain protecting groups must be stable to the conditions used to remove the temporary α-amino protecting group prior to the next coupling step.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

Compounds for use in accordance with the present invention (e.g. LTX-109) may be synthesized as described in WO 2009/081152A2.

Compounds of use in accordance with the present invention exhibit activity against causative agents of sinusitis, pneumonia and/or otitis. Certain pathogens are causative agents of sinusitis, pneumonia and/or otitis. Such pathogens (which may also be referred to as infectious agents) include bacteria, viruses and fungi.

Compounds of use in the present invention typically exhibit activity against viruses (antiviral activity) in (or as determined by or as assessed by) a suitable in vitro assay, for example an endpoint dilution assay (e.g. a TCID50 assay). The skilled person is familiar with suitable in vitro assays, for example suitable endpoint dilution assays (e.g. TCID50 assays). Preferred TCID50 assays are described in the Example section herein. Compounds of use in the present invention typically exhibit activity against bacteria and/or fungi (antibacterial and/or antifungal activity) in (or as determined by or as assessed by) a suitable in vitro assay, e.g. an assay to determine minimal inhibitory concentration (MIC) of the compound against a given organism, for example a microbroth dilution method (e.g. as described in the Example section herein).

As indicated above, the present invention provides compounds as defined elsewhere herein for use in treating sinusitis, pneumonia and/or otitis. The present invention provides a compound as defined herein for use in treating an infection that causes (or is associated with or is characteristic of) sinusitis, pneumonia or otitis in a subject having, or suspected of having, sinusitis, pneumonia or otitis.

The causative agent of sinusitis, pneumonia or otitis may be a bacteria, a virus and/or a fungus. Thus, the sinusitis, pneumonia or otitis may be viral sinusitis, pneumonia or otitis, bacterial sinusitis, pneumonia or otitis, or fungal sinusitis, pneumonia or otitis. In some cases there may be more than one type of causative agent (infectious agent) present. Thus, in some embodiments, the sinusitis, pneumonia or otitis may be polymicrobial sinusitis, pneumonia or otitis. In some embodiments, the sinusitis, pneumonia or otitis may be caused by at least a viral causative agent, and optionally a bacterial causative agent and/or a fungal causative agent may additionally be present.

In some embodiments, the causative agent of sinusitis, pneumonia or otitis is a bacteria, or is suspected to be a bacteria.

In some embodiments, the bacteria may be a bacteria of the genus Haemophilus, Streptococcus, Moraxella, Pseudomonas or Mycoplasma. In some embodiments, the bacteria may be Haemophilus influenzae, Streptococcus pneumoniae, Streptococcus pyogenes, Moraxella catarrhalis, Pseudomonas aeruginosa or Mycoplasma pneumoniae.

Haemophilus influenzae, Streptococcus pneumonia and Streptococcus pyogenes can cause sinusitis, pneumonia and otitis. Moraxella catarrhalis can cause sinusitis and otitis. Pseudomonas aeruginosa and Mycoplasma pneumoniae can cause pneumonia. Pseudomonas aeruginosa can cause otitis.

In some embodiments, the causative agent of sinusitis, pneumonia or otitis is a virus (for example an enveloped or non-enveloped (e.g. icosahedral) virus), or is suspected to be a virus (for example an enveloped or non-enveloped (e.g. icosahedral) virus).

In some embodiments, the virus may be an Orthopneumovirus (e.g. Respiratory Syncytial Virus, RSV), an Orthomyxovirus (e.g. the Influenza A virus), from the genus Enterovirus (e.g. a Rhinovirus) or a Coronavirus (e.g. the Severe Acute Respiratory Syndrome Coronavirus 2, SARS-CoV-2).

In some embodiments, the virus may be selected from the group consisting of Respiratory Syncytial Virus (RSV), Influenza A virus, Rhinovirus and Severe Acute Respiratory Syndrome Coronavirus 2 (SARS-CoV-2).

In some embodiments, a virus from the genus Enterovirus may be enterovirus C (sometimes also referred to as enterovirus species C or a type C enterovirus) or enterovirus D (sometimes referred to as enterovirus species D or a type D enterovirus). In some embodiments, the enterovirus C may be enterovirus C104 (EV-C104), enterovirus C105 (EV-C105), enterovirus C109 (EV-C109), enterovirus C117 (EV-C117), or enterovirus C118 (EV-C118). In some embodiments, the enterovirus D may be enterovirus D68 (EV-D68).

Influenza A virus, Rhinovirus and RSV can cause sinusitis, pneumonia and otitis. SARS-CoV-2 can cause pneumonia. Enterovirus C and Enterovirus D (e.g. enterovirus D68) can cause pneumonia.

In some embodiments, the causative agent of sinusitis, pneumonia or otitis is a fungus, or is suspected to be a fungus.

In some embodiments, the fungus may be a fungus of the genus Candida or Aspergillus. In some embodiments, the fungus may be selected from the group consisting of Candida albicans, Candida tropicalis, Candida parapsilosis, Candida krusei and Aspergillus niger. These fungi can cause sinusitis, pneumonia and otitis.

In some embodiments, the sinusitis, pneumonia or otitis may be polymicrobial, i.e. there may be more than one type of pathogen present (e.g. virus and bacteria, or virus and fungus, or bacteria and fungi, or virus and bacteria and fungi).

Typically and preferably of course, the causative agent (or pathogen) is a causative agent that infects (or is capable of infecting) a mammal. Mammals include, for example, humans and any livestock, domestic or laboratory animal. Specific examples include mice, rats, pigs, cats, dogs, sheep, rabbits, cows and monkeys. In some embodiments of the present invention the mammal is a human. Thus, typically and preferably, the causative agent of sinusitis, pneumonia or otitis in accordance with the present invention is mammalian pathogen, preferably a human pathogen.

In some embodiments, compounds as defined herein are for use in treating sinusitis.

In some embodiments, compounds as defined herein are for use in treating pneumonia.

In some embodiments, compounds as defined herein are for use in treating otitis. Otitis may be otitis media or otitis externa. In some preferred embodiments of the present invention, the otitis is otitis media. In some other preferred embodiments of the present invention, the otitis is otitis externa.

In some embodiments, compounds as defined herein are for use in treating more than one condition selected from the group consisting of sinusitis, pneumonia and otitis in a given subject.

In some embodiments, the causative agent, or the type of causative agent, of the sinusitis, pneumonia or otitis in the subject is known (or is determined or has been determined or is diagnosed or has been diagnosed). Thus, in some embodiments it is known whether the causative agent is a virus, a bacteria or a fungus (or a combination thereof). In some embodiments, the causative agent, or the type of causative agent, of the sinusitis, pneumonia or otitis in the subject is known (or is determined, or has been determined, or is diagnosed, or has been diagnosed) prior to administration of the compound to the subject, for example prior to the first administration of the compound to the subject.

However, in other embodiments, the causative agent, or the type of causative agent (bacteria, virus or fungus), of the sinusitis, pneumonia or otitis in the subject is not known (or is not determined or has not been determined or is not diagnosed or has not been diagnosed). Bacteria, viruses and fungi are types of causative agent (infectious agent), thus if a “type of” causative agent is not known then it is not known if the causative agent is a bacteria, virus or fungus (or a combination thereof). A “causative agent” can be alternatively viewed as an “infectious agent”, and a “type of causative agent” can be alternatively viewed as a “type of infectious agent”.

Thus, in some embodiments it is not known whether the causative agent is a virus, a bacteria or a fungus (or a combination thereof). In some embodiments, the causative agent, or the type of causative agent, of the sinusitis, pneumonia or otitis in the subject is not known (or is not determined, or has not been determined, or is not diagnosed, or has not been diagnosed) prior to administration of the compound to the subject, for example prior to the first administration of the compound to the subject.

As described above, the present inventors have found that compounds of use in the present invention exhibit excellent antiviral activity against viruses that are causative agents of sinusitis, pneumonia and otitis, in addition to exhibiting excellent activity against bacteria and fungi that are causative agents of sinusitis, pneumonia and otitis. This surprising finding is advantageous, as sinusitis, pneumonia and otitis can be treated with compounds as defined herein without needing to know the underlying causative agent, or the type of causative agent, of the disease. Such compounds would be effective in the treatment of sinusitis, pneumonia and otitis irrespective of whether the cause of the disease is a bacterial infection, a viral infection, or a fungal infection (or a polymicrobial infection).

Thus, in some embodiments of the present invention, compounds of use in the present invention may be administered to the subject without knowledge of the causative agent, or the type of causative agent, of the sinusitis, pneumonia or otitis. Compounds of use in the present invention may be administered to the subject without knowledge of the causative agent, or the type of causative agent, of the sinusitis, pneumonia or otitis prior to administration of the compound to the subject, for example prior to the first administration of the compound to the subject.

In some embodiments of the present invention, there has been no determination made (or no definitive determination made) of the causative agent, or the type of causative agent, of the sinusitis, pneumonia or otitis. In some embodiments, there has been no determination made (or no definitive determination made) of the causative agent, or the type of causative agent, of the sinusitis, pneumonia or otitis prior to administration of the compound to the subject, for example prior to the first administration of the compound to the subject.

In some embodiments of the present invention, no diagnosis (or no definitive diagnosis) of the causative agent, or the type of causative agent, of the sinusitis, pneumonia or otitis is made or has been made. In some embodiments, no diagnosis (or no definitive diagnosis) is made or has been made of the causative agent, or the type of causative agent, of the sinusitis, pneumonia or otitis prior to administration of the compound to the subject, for example prior to the first administration of the compound to the subject.

Thus, in some embodiments, a determination or diagnosis (e.g. a definitive determination or definitive diagnosis) of the causative agent, or the type of causative agent, of the sinusitis, pneumonia or otitis is not made or has not been made. In some embodiments, a determination (or diagnosis) of the causative agent, or the type of causative agent, of the sinusitis, pneumonia or otitis is not made, or a determination (or diagnosis) of the causative agent sinusitis, pneumonia or otitis has not been made prior to administration of the compound to the subject, for example prior to the first administration of the compound to the subject.

In some embodiments, there is no performance of tests or assays (e.g. laboratory tests or assays) to determine or establish (or no step of determining or establishing) the causative agent, or the type of causative agent, of the sinusitis, pneumonia or otitis. In some embodiments, there is no performance of tests or assays (e.g. laboratory tests) to determine the causative agent, or the type of causative agent, of the sinusitis, pneumonia or otitis prior to administration of the compound to the subject, for example prior to the first administration of the compound to the subject.

In one aspect, the present invention provides a compound as defined elsewhere herein for use in the treatment of an infection in a subject having (or suspected of having) sinusitis, pneumonia or otitis wherein the infectious agent, or the type of infectious agent, of (or causing) the sinusitis, pneumonia or otitis has not been determined. In some embodiments, the infectious agent, or the type of infectious agent, causing the sinusitis, pneumonia or otitis is not determined (or has not been determined) prior to administration of the compound to the subject, for example prior to the first administration of the compound to the subject. Embodiments of the invention described herein in relation to other aspects of the invention apply, mutatis mutandis, to this aspect of the invention.

In one aspect, the present invention provides a compound as defined elsewhere herein for use in the treatment of an infection in a subject having (or suspected of having) sinusitis, pneumonia or otitis wherein the infectious agent, or the type of infectious agent, of (or causing) the sinusitis, pneumonia or otitis is not known. In some embodiments, the infectious agent, or the type of infectious agent, causing the sinusitis, pneumonia or otitis is not known prior to administration of the compound to the subject, for example prior to the first administration of the compound to the subject. Embodiments of the invention described herein in relation to other aspects of the invention apply, mutatis mutandis, to this aspect of the invention.

In one aspect, the present invention provides a compound as defined elsewhere herein for use in the treatment of an infection in a subject having (or suspected of having) sinusitis, pneumonia or otitis irrespective of the type of infectious agent. Thus, the present invention provides a compound as defined elsewhere herein for use in the treatment of an infection in a subject having (or suspected of having) sinusitis, pneumonia or otitis irrespective of whether the infectious agent is a bacteria, a virus or a fungus (or a combination thereof). Embodiments of the invention described herein in relation to other aspects of the invention apply, mutatis mutandis, to this aspect of the invention.

For the avoidance of doubt, references to aspects or embodiments of the invention in which the causative agent (or infectious agent) “is not known” or “is not determined” or “has not been determined” (or similar) means that the causative agent (or infectious agent) “is not known” or “is not determined” or “has not been determined” by the subject itself or by any person involved in the treatment of the subject (e.g. a clinician or other healthcare personnel). Conversely, references to embodiments of the invention in which the causative agent (or infectious agent) “is known” or “is determined” or “has been determined” (or similar) means that the causative agent (or infectious agent) “is known” or “is determined” or “has been determined” by the subject itself or by any person involved in the treatment of the subject (e.g. a clinician or other healthcare personnel).

Compounds for use in accordance with the invention are typically presented (or administered) in the form of a formulation (for example a solution) or composition comprising one or more compounds in accordance with the invention in admixture with a suitable diluent, carrier and/or excipient. Suitable diluents, excipients and carriers are known to the skilled person. Thus, the invention provides a formulation (or composition) comprising a compound as defined herein for use in treating sinusitis, pneumonia or otitis. Typically and preferably of course, the formulation (or composition) is a pharmaceutical formulation (or pharmaceutical composition). Thus, preferably diluents, carriers and/or excipients are pharmaceutically acceptable diluents carriers and/or carriers.

The compositions for use according to the invention may be presented, for example, in a form suitable for oral, nasal, respiratory tract (e.g. upper respiratory tract or lungs), auricular (or otic), parenteral, intravenal or topical administration. The skilled person is readily able to select an appropriate form for administration, for example based on the condition to be treated.

The compounds (or formulations or compositions) for use in accordance with the invention may be administered orally, nasally, auricularly (or oticly, i.e. to or by way of the ear), parenterally, intravenously, topically, or by inhalation.

The compounds (or formulations or compositions) for use in accordance with the invention may be administered to the respiratory tract, e.g. the upper respiratory tract. The compounds (or formulations or compositions) for use in accordance with the invention may be administered to the lungs.

In some embodiments, the compounds (or formulations or compositions) for use in accordance with the invention may be administered to the upper respiratory tract, for example the administration may be nasal administration (e.g. with a nasal spray). An upper respiratory tract, preferably nasal, administration route may be preferred when the condition to be treated is sinusitis.

In some embodiments, nasal administration of compounds (or formulations or compositions) may be via (or performed using) a nasal spray.

In some embodiments, nasal administration of compounds (or formulations or compositions) may be via (or performed using) any device or delivery system suitable for nasal administration. For example nasal administration may be via (or performed using) a vapour inhaler, a mechanical spray pump (e.g. a bi-directional multi-dose spray pump, e.g. OptiNose), a gas driven spray system/atomizer, a nebulizer, an electrically powered nebulizer/atomizer, a mechanical powder sprayer, a breath actuated nasal inhaler or an insufflator (e.g. a breath powered bi-directional delivery insufflator (e.g. OptiNose)).

In some embodiments, nasal administration may be via (or performed using) drops (nose drops).

In some embodiments, the condition to be treated is sinusitis and the compounds (or formulations or compositions) are administered nasally, preferably with a nasal spray.

In some embodiments, the compounds (or formulations or compositions) for use in accordance with the invention may be administered to the respiratory tract (e.g. to the lungs). Such administration may be, for example, by inhalation (e.g. with an inhaler such as a metered dose inhaler, or a nebulizer, or the like). Thus, the compounds (or formulations or compositions) may be inhaled. A respiratory tract administration route, for example by inhalation, may be preferred when the condition to be treated is pneumonia. In some embodiments, administration of compounds (or formulations or compositions) by way of inhalation may be via (or performed using) an inhaler or nebulizer or the like. In some embodiments, the condition to be treated is pneumonia and the compounds (or formulations or compositions) are administered by inhalation.

In some embodiments, the compounds (or formulations or compositions) for use in accordance with the invention may be administered by auricular (or otic) administration (i.e. administration to, or by way of, the ear). An auricular administration route may be preferred when the condition to be treated is otitis (e.g. otitis media which is an infection of the middle ear, or otitis externa which is an infection of the external ear canal). In some embodiments, auricular (or otic) administration of compounds (or formulations or compositions) may via (or performed using) using ear drops or the like, or by intratympanic injection. Ear drops may be preferred. In some embodiments, the condition to be treated is otitis and the compounds (or formulations or compositions) are administered by otic administration.

As used herein, the term “pharmaceutical” includes veterinary applications of the invention.

The active compounds defined herein may be presented in the conventional pharmacological forms of administration, such as tablets, coated tablets, nasal sprays, ear drops, solutions, emulsions, liposomes, powders, capsules, or sustained release forms, or forms suitable for inhalation.

Conventional pharmaceutical excipients as well as the usual methods of production may be employed for the preparation of these forms.

Tablets may be produced, for example, by mixing the active ingredient or ingredients with known excipients, such as for example with diluents, such as calcium carbonate, calcium phosphate or lactose, disintegrants such as corn starch or alginic acid, binders such as starch or gelatin, lubricants such as magnesium stearate or talcum, and/or agents for obtaining sustained release, such as carboxypolymethylene, carboxymethyl cellulose, cellulose acetate phthalate, or polyvinylacetate.

The tablets may if desired consist of several layers. Coated tablets may be produced by coating cores, obtained in a similar manner to the tablets, with agents commonly used for tablet coatings, for example, polyvinyl pyrrolidone or shellac, gum arabic, talcum, titanium dioxide or sugar. In order to obtain sustained release or to avoid incompatibilities, the core may consist of several layers too. The tablet-coat may also consist of several layers in order to obtain sustained release, in which case the excipients mentioned above for tablets may be used.

Solutions (e.g. injection solutions) may, for example, be produced in the conventional manner, such as by the addition of preservation agents, such as p-hydroxybenzoates, or stabilizers, such as EDTA. The solutions may be filled into vials or ampoules or into devices to be used for administration (e.g. nasal spray devices, inhalers, nebulizers, ear drop bottles, or the like).

Nasal sprays may be formulated in solution (e.g. aqueous solution) and packed into spray containers either with an aerosol propellant or provided with means for manual compression.

Capsules containing one or several active ingredients may be produced, for example, by mixing the active ingredients with inert carriers, such as lactose or sorbitol, and filling the mixture into gelatin capsules.

Dosages may vary based on parameters such as the age, weight and sex of the subject. Appropriate dosages can be readily established by the skilled person. Appropriate dosage units can readily be prepared.

Treatments in accordance with the present invention may involve co-administration with one or more further active agent that is used in the treatment or prevention of sinusitis, pneumonia or otitis. Speaking generally, the one or more further active agent may be administered to the subject substantially simultaneously with the compound in accordance with the invention; such as from a single pharmaceutical composition or from two pharmaceutical compositions administered closely together. Thus, in some embodiments, pharmaceutical compositions may additionally comprise one or more further active ingredients. Alternatively, one or more further active agent may be administered to the subject at a time sequential to the administration of a compound in accordance with the invention. “At a time sequential”, as used herein, means “staggered”, such that the one or more further agent is administered to the subject at a time distinct to the administration of the compound in accordance with the invention. Generally, the two agents would be administered at times effectively spaced apart to allow the two agents to exert their respective therapeutic effects, i.e., they are administered at “biologically effective time intervals”. The one or more further active agent may be administered to the subject at a biologically effective time prior to the compound in accordance with the invention, or at a biologically effective time subsequent to the compound in accordance with the invention.

In one aspect, the present invention provides a compound as defined elsewhere herein for use in the treatment of viral, bacterial and/or fungal sinusitis, viral, bacterial and/or fungal pneumonia, or viral, bacterial and/or fungal otitis in a subject. In some embodiments, the compound is for use in the treatment of viral sinusitis, pneumonia or otitis in a subject. Embodiments of the invention described herein in relation to other aspects of the invention apply, mutatis mutandis, to this aspect of the invention. For example, in some embodiments, the type of causative agent of the sinusitis, pneumonia or otitis is not known prior to the first administration of said compound to said subject.

In another aspect, the present invention provides a compound as defined elsewhere herein for use in the treatment of sinusitis, pneumonia or otitis that is caused by a viral, bacterial and/or fungal infection in a subject. In some embodiments, the compound is for use in the treatment of sinusitis, pneumonia or otitis that is caused by a viral infection. Embodiments of the invention described herein in relation to other aspects of the invention apply, mutatis mutandis, to this aspect of the invention. For example, in some embodiments, the type of causative agent of the sinusitis, pneumonia or otitis is not known prior to the first administration of said compound to said subject.

In another aspect, the present invention provides a compound as defined elsewhere herein for use in treating an infection of the sinuses in a subject having or suspected of having sinusitis, or for use in treating an infection of the lungs in a subject having or suspected of having pneumonia, or for use in treating an infection of the ear in a subject having or suspected of having otitis (e.g. an infection of the middle ear in the case of otitis media, or an infection of the external ear canal in the case of otitis externa). The infection may be a viral infection, or a bacterial infection, or a fungal infection, or a polymicrobial infection. In some embodiments, the infection may be a viral infection. Embodiments of the invention described herein in relation to other aspects of the invention apply, mutatis mutandis, to this aspect of the invention. For example, in some embodiments, the type of causative agent of the sinusitis, pneumonia or otitis is not known prior to the first administration of said compound to said subject.

In another aspect, the invention provides a compound as defined elsewhere herein for use in treating an infection that causes (or is causing or has caused) sinusitis, pneumonia or otitis in a subject having, or suspected of having, sinusitis, pneumonia or otitis. The infection may be a viral infection, or a bacterial infection, or a fungal infection, or a polymicrobial infection. In some embodiments, the infection may be a viral infection. In some embodiments, the infection may be a polymicrobial infection (e.g. a polymicrobial infection as discussed elsewhere herein). In some embodiments, the infection may be a polymicrobial infection in which at least one of the pathogens is, or is suspected to be, a virus. Embodiments of the invention described herein in relation to other aspects of the invention apply, mutatis mutandis, to this aspect of the invention. For example, in some embodiments, the type of causative agent of the sinusitis, pneumonia or otitis is not known prior to the first administration of said compound to said subject.

In another aspect, the present invention provides a compound as defined elsewhere herein for use in treating an infection that has caused (or is causing) sinusitis, pneumonia or otitis in a subject. The infection may be a viral infection, or a bacterial infection, or a fungal infection, or a polymicrobial infection. In some embodiments, the infection may be a viral infection. Embodiments of the invention described herein in relation to other aspects of the invention apply, mutatis mutandis, to this aspect of the invention. For example, in some embodiments, the type of causative agent of the sinusitis, pneumonia or otitis is not known prior to the first administration of said compound to said subject.

In another aspect, the present invention provides a compound as defined elsewhere herein for use in treating sinusitis, pneumonia or otitis in a subject, wherein said sinusitis, pneumonia or otitis is, or is potentially, or is suspected of being, caused by (or associated with or characterised by) at least a viral infection. Embodiments of the invention described herein in relation to other aspects of the invention apply, mutatis mutandis, to this aspect of the invention.

In another aspect, the present invention provides a compound as defined elsewhere herein for use in treating sinusitis, pneumonia or otitis in a subject, wherein said sinusitis, pneumonia or otitis is, or is potentially, or is suspected of being, caused by (or associated with or characterised by) at least a bacterial infection. Embodiments of the invention described herein in relation to other aspects of the invention apply, mutatis mutandis, to this aspect of the invention.

In another aspect, the present invention provides a compound as defined elsewhere herein for use in treating sinusitis, pneumonia or otitis in a subject, wherein said sinusitis, pneumonia or otitis is, or is potentially, or is suspected of being, caused by (or associated with or characterised by) at least a fungal infection. Embodiments of the invention described herein in relation to other aspects of the invention apply, mutatis mutandis, to this aspect of the invention.

In another aspect, the invention provides a compound as defined elsewhere herein for use in treating an infection that is associated with sinusitis, pneumonia or otitis in a subject having, or suspected of having, sinusitis, pneumonia or otitis. The infection may be a viral infection, or a bacterial infection, or a fungal infection, or a polymicrobial infection. Embodiments of the invention described herein in relation to other aspects of the invention apply, mutatis mutandis, to this aspect of the invention. For example, in some embodiments, the type of infectious agent associated with the sinusitis, pneumonia or otitis is not known prior to the first administration of said compound to said subject.

The term “treatment” or “therapy” used herein includes therapeutic and preventative (or prophylactic) therapies. Thus, compounds for use in accordance with the invention may be for therapeutic or prophylactic uses. “Treatment” or “therapy” includes administration of a compound in accordance with the invention to subjects having, or suspected of having, sinusitis, pneumonia or otitis.

Alternatively viewed, the present invention provides a method of treating sinusitis, pneumonia or otitis in a subject (or patient) which method comprises administering to a subject in need thereof a therapeutically or prophylactically effective amount of a compound as defined herein. Embodiments of the invention described herein in relation to other aspects of the invention apply, mutatis mutandis, to this aspect of the invention. For example, in some embodiments, the type of causative agent of the sinusitis, pneumonia or otitis is not known prior to the first administration of said compound to said subject.

An effective amount (e.g. therapeutically or prophylactically effective amount) will be determined based on the clinical assessment and can be readily monitored. An amount administered should typically be effective to kill or inactivate all or a proportion of the causative agent or to prevent or reduce their rate of reproduction or otherwise to lessen their harmful effect on the body. Administration may also be prophylactic.

Further alternatively viewed, the present invention provides the use of a compound as defined herein in the manufacture of a medicament for use in the treatment of sinusitis, pneumonia or otitis. Embodiments of the invention described herein in relation to other aspects of the invention apply, mutatis mutandis, to this aspect of the invention. For example, in some embodiments, the type of causative agent of the sinusitis, pneumonia or otitis is not known prior to the first administration of said compound to said subject. Thus, in some embodiments of uses for manufacture of a medicament in accordance with the invention, the treatment comprises administering to a subject in need thereof an effective amount of a compound in accordance with the invention, wherein the type of causative agent causing the sinusitis, pneumonia or otitis is not known prior to the first administration of said compound to said subject.

Further alternatively viewed, the present invention provides the use of a compound as defined herein for the treatment of sinusitis, pneumonia or otitis. Embodiments of the invention described herein in relation to other aspects of the invention apply, mutatis mutandis, to this aspect of the invention. For example, in some embodiments, the causative agent of the sinusitis, pneumonia or otitis is not known prior to the first administration of said compound to said subject.

The term “subject” or “patient” as used herein includes any mammal, for example humans and any livestock, domestic or laboratory animal. Specific examples include mice, rats, pigs, cats, dogs, sheep, rabbits, cows and monkeys. Preferably, however, the subject or patient is a human subject. Thus, subjects or patients treated in accordance with the present invention will preferably be humans.

In some embodiments, subjects in accordance with the present invention are subjects having sinusitis, pneumonia or otitis. In some embodiments, subjects in accordance with the present invention are subjects suspected of having sinusitis, pneumonia or otitis. In some embodiments, subjects in accordance with the present invention may be subjects at risk of developing (or at risk of contracting) sinusitis, pneumonia or otitis.

The invention also provides kits comprising one or more of the compounds in accordance with the invention for use in the methods and uses described herein. Preferably said kits comprise instructions for use in treating sinusitis, pneumonia or otitis as described herein.

As used throughout the entire application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms.

The invention will now be further described with reference to the following non-limiting Examples.

### EXAMPLES

### Example 1: Antiviral Activity of LTX-109 Against Influenza A Virus (IAV)

Aim

The aim of this study was to test the antiviral activity of LTX-109 against IAV (Influenza A virus).

Methods

To test whether 1% LTX-109 (w/v) has antiviral activity against IAV, 1×10⁶ infectious units of IAV (A/WSN/33; 40 μl) were incubated with four volumes of 1% LTX-109 dissolved in PBS (160 μl) or a PBS (Phosphate-buffered saline) control. The experiment was performed in triplicates.

After 1 hour, the incubation was stopped by adding an excess of cold media, and the formulation was physically separated from the virus through a filter to reduce cytotoxicity on the assay cells. Some cloudiness in the preparation was observed for the 1% formulation, but no precipitation. Infectious virus was quantified through a serial dilution (a series of ten-fold dilutions) on monolayers of MDCK-II cells in microtitre plates (MDCK-II cells are mammalian cells capable of displaying a cytopathic effect (CPE) upon viral infection). The starting solution for the serial dilution (i.e. the neat (or undiluted) solution) was obtained by re-suspending the virus that was separated via the filtration step in 1 ml of media. For each dilution of the virus in the dilution series, eight wells of the microtitre plate were tested (i.e. each dilution of virus was applied to eight separate wells, each well containing a MDCK-II cell monolayer). Appropriate controls were also performed. Five days after infection of the cells, virus titre was quantified by determining the dilution at which half of the cells (half of the wells at a given dilution) displayed virus-induced cytopathic effect (TCID50). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to/ml of the starting solution (i.e. neat/undiluted solution) mentioned above.

A parallel test where the same procedure was carried out in the absence of virus was included to determine any residual cytotoxic effect of the formulation on the assay cells.

Results

The results of the test for antiviral activity of 1% LTX-109 against Influenza A virus (IAV) are summarized in Table 1 (below).

After incubation with the PBS control for 1 h an average of 5.16 E+04 TCID50/ml of IAV was measured.

After 1 h incubation with 1% LTX-109, an average of 1.58 E+01 TCID50/ml was measured, which corresponds to a decrease in infectivity of over 3 logs, or 99.9%, as compared to the PBS control.

After filtration, cytotoxicity was observed only up to the first dilution of the 1% formulation, without affecting the validity of the test.

TABLE 1
Average virus titres recovered after incubation with PBS or 1% LTX 109 for 1 h.
A decrease of over 3 log of infectivity compared to the PBS control was measured.

| | | | | Log | |
|---|---|---|---|---|---|
| | With Filtration | TCID50/ml ± SEM | Log | decrease | Percentage |
| PBS 1 h | No cytotoxicity | 5.16E+04 ± 2.11E+04 | 4.62 | | |
| 1% LTX- | Up to 10\hat{ }−1 | 1.58E+01 ± 0.00E+00 | 1.20 | 3.42 | 99.969 |
| 109 1 h | (i.e. cytotoxicity | | | | |
| | only up to the | | | | |
| | 10⁻¹ dilution) | | | | |

### Conclusions

Based on the findings reported here, exposure of IAV to 1% LTX-109 for 1 h in vitro caused over 3 logs decrease in virus infectivity as compared to the PBS control, which corresponds to a 99.9% reduction. These results show that LTX-109 has excellent antiviral activity against Influenza A virus. Influenza A is a causative agent of sinusitis, pneumonia and otitis media.

### Example 2: Antiviral Activity of LTX-109 Against Respiratory Syncytial Virus (RSV)

Aim

The aim of this study was to test the antiviral activity of LTX-109 against RSV (Respiratory Syncytial Virus).

Methods

To test whether 1% LTX-109 (w/v) and 0.1% LTX-109 (w/v) have antiviral activity against RSV, 1×10⁵ infectious units of RSV (40 μl) were incubated with four volumes of 1% LTX-109 or 0.1% LTX-109 dissolved in PBS (160 μl) or a PBS (Phosphate-buffered saline) control. The experiment was performed in triplicates.

After 1 hour, the incubation was stopped by adding an excess of cold media, and the formulation was physically separated from the virus through a filter to reduce cytotoxicity on the assay cells. Some cloudiness in the preparation was observed, particularly for the 1% formulation, but no precipitation. Infectious virus was quantified through a serial dilution (a series of ten-fold dilutions) on monolayers of Hep2 cells in microtitre plates (Hep2 cells are mammalian cells capable of displaying a cytopathic effect (CPE) upon viral infection). The starting solution for the serial dilution (i.e. the neat (or undiluted) solution) was obtained by re-suspending the virus that was separated via the filtration step in 1 ml of media. For each dilution of the virus in the dilution series, eight wells of the microtitre plate were tested (i.e. each dilution of virus was applied to eight separate wells, each well containing a Hep2 cell monolayer). Appropriate controls were also performed. Eight days after infection of the cells, virus titre was quantified by determining the dilution at which half of the cells (half of the wells at a given dilution) displayed virus-induced cytopathic effect (TCID50). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to/ml of the starting solution (i.e. neat/undiluted solution) mentioned above.

A parallel test where the same procedure was carried out in the absence of virus was included to determine any residual cytotoxic effect of the formulation on the assay cells.

Results

The results of the test for antiviral activity of 1% LTX-109 and 0.1% LTX-109 against Respiratory Syncytial Virus (RSV) are summarized in Table 2 (below).

After incubation with the PBS control for 1 h an average of 3.13 E+05 TCID50/ml of RSV was measured.

After 1 h incubation with 1% LTX-109, an average of 1.58 E+02 TCID50/ml was measured, which corresponds to a decrease in infectivity of over 3 logs, or 99.9%, as compared to the PBS control.

After 1 h incubation with 0.1% LTX-109, an average of 8.76 E+01 TCID50/ml was measured, which corresponds to a decrease in infectivity of over 3 logs, or 99.9%, as compared to the PBS control.

After filtration, cytotoxicity was observed only up to the first dilution of the 1% formulation, without affecting the validity of the test. No cytotoxicity was observed for the 0.1% formulation.

TABLE 2
Average virus titres recovered after incubation with PBS or 1% LTX-109 or
0.1% LTX-109 for 1 h. A decrease of over 3 log of infectivity compared to
the PBS control was measured for both of the LTX-109 concentrations tested.

| | | | | Log | |
|---|---|---|---|---|---|
| | With Filtration | TCID50/ml ± SEM | Log | decrease | Percentage |
| PBS 1 h | No cytotoxicity | 3.13E+05 ± 1.00E+05 | 5.45 | | |
| 1% LTX- | Up to 10\hat{ }−1 | 1.58E+02 ± 0.00E+00 | 2.20 | 3.25 | 99.949 |
| 109 1 h | (i.e. cytotoxicity | | | | |
| | only up to the | | | | |
| | 10⁻¹ dilution) | | | | |
| 0.1% LTX- | No cytotoxicity | 8.76E+01 ± 4.11E+01 | 1.78 | 3.67 | 99.97 |
| 109 1 h | | | | | |

### Conclusions

Based on the findings reported here, exposure of RSV to 1% LTX-109 or 0.1% LTX-109 for 1 h in vitro caused over 3 logs decrease in virus infectivity as compared to the PBS control, which corresponds to a 99.9% reduction. These results show that LTX-109 has excellent antiviral activity against RSV. RSV is a causative agent of sinusitis, pneumonia and otitis media.

### Example 3: Antiviral Activity of LTX-109 Against the SARS-CoV-2 Virus

Aim

The aim of this study was to test the antiviral activity of LTX-109 against the SARS-CoV-2 virus.

Methods

The SARS-CoV-2 used was from BEI Resources: SARS-CoV-2 Isolate England02/2020 (Catalogue No. NR-52359).

To test whether 1% LTX-109 (w/v) has antiviral activity against SARS-CoV-2, 5×10⁶ infectious units of SARS-CoV2 (40 μl) were incubated with four volumes of 1% LTX-109 dissolved in PBS (160 μl) or a PBS (Phosphate-buffered saline) control. The experiment was performed in triplicates.

After 1 hour, the incubation was stopped by adding an excess of cold media, and the formulation was physically separated from the virus through a filter to reduce cytotoxicity on the assay cells. Some cloudiness in the preparation was observed for the 1% formulation, but no precipitation. Infectious virus was quantified through a serial dilution (a series of ten-fold dilutions) on monolayers of Vero cells in microtitre plates (Vero cells are mammalian cells capable of displaying a cytopathic effect (CPE) upon viral infection). The starting solution for the serial dilution (i.e. the neat (or undiluted) solution) was obtained by re-suspending the virus that was separated via the filtration step in 1 ml of media. For each dilution of the virus in the dilution series, eight wells of the microtitre plate were tested (i.e. each dilution of virus was applied to eight separate wells, each well containing a Vero cell monolayer). Appropriate controls were also performed. Five days after infection of the cells, virus titre was quantified by determining the dilution at which half of the cells (half of the wells at a given dilution) displayed virus-induced cytopathic effect (TCID50). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to/ml of the starting solution (i.e. neat/undiluted solution) mentioned above.

A parallel test where the same procedure was carried out in the absence of virus was included to determine any residual cytotoxic effect of the formulation on the assay cells.

Results

The results of the test for antiviral activity of 1% LTX-109 against the SARS-CoV-2 virus are summarized in Table 3 (below).

After incubation with the PBS control for 1 h an average of 4.27 E+06 TCID50/ml of SARS-CoV-2 was measured.

After 1 h incubation with 1% LTX-109, an average of 1.99 E+02 TCID50/ml was measured, corresponding to a decrease in infectivity of over 4 logs, or 99.99%, as compared to the PBS control.

After filtration, cytotoxicity was observed only up to the first dilution of the 1% formulation, without affecting the validity of the test.

TABLE 3
Average virus titres recovered after incubation with PBS or 1% LTX-109 for 1 h.
A decrease of over 4 log of infectivity compared to the PBS control was measured

| | | | | Log | |
|---|---|---|---|---|---|
| | With Filtration | TCID50/ml ± SEM | Log | decrease | Percentage |
| PBS 1 h | No cytotoxicity | 4.27E+06 ± 7.30E+05 | 6.62 | | |
| 1% LTX | Up to 10\hat{ }−1 | 1.99E+02 ± 4.11E+01 | 2.28 | 4.33 | 99.995 |
| 109 1 h | (i.e. cytotoxicity | | | | |
| | only up to the | | | | |
| | 10⁻¹ dilution) | | | | |

### Conclusions

Based on the findings reported here, exposure of SARS-CoV-2 to 1% LTX-109 for 1 h in vitro caused over 4 logs decrease in virus infectivity as compared to the PBS control, which corresponds to a 99.99% reduction. These results show that LTX-109 has excellent antiviral activity against SARS-CoV-2. SARS-CoV-2 is a causative agent of pneumonia.

### Example 4: Antibacterial and Antifungal Activity of LTX-109

This Example describes antibacterial activity and antifungal activity of the compound LTX-109 against certain species of bacteria (both Gram-positive and Gram-negative bacteria) and fungi that can cause sinusitis, pneumonia and/or otitis.

Activity of LTX-109 against the following organisms is described: Haemophilus influenzae, Streptococcus pneumoniae, Streptococcus pyogenes, Moraxella catarrhalis, Pseudomonas aeruginosa, Candida albicans, Candida tropicalis, Candida parapsilosis, Candida krusei and Aspergillus niger.

Haemophilus influenzae, Streptococcus pneumonia and Streptococcus pyogenes can cause sinusitis, pneumonia and otitis media. Moraxella catarrhalis can cause sinusitis and otitis media. Pseudomonas aeruginosa can cause pneumonia and otitis externa. The fungi Candida and Aspergillus niger can also cause sinusitis, pneumonia, otitis media and otitis externa.

Materials and Methods

The compound LTX-109 has the structural formula as set out elsewhere herein.

Bacterial and fungal isolates used in this study were from various sources worldwide stored at GR Micro Ltd. and maintained, with minimal sub-culture, deep frozen at −70° C. as a dense suspension in a high protein matrix of undiluted horse serum. The species used and their characteristics are listed in Table 4 below.

Minimum inhibitory concentration (MIC) of LTX-109 was determined using the following microbroth dilution methods for antimicrobial susceptibility testing published by the Clinical and Laboratory Standards Institute (CLSI, formerly NCCLS):
M7-A6 Vol. 23 No. 2 Jan. 2003 Methods for Dilution Antimicrobial Susceptibility Tests for Bacteria that Grow Aerobically; Approved Standard-Sixth Edition. National Committee for Clinical Laboratory Standards.
M100-S15 Vol. 25 No 1. January 2005 Performance Standards for Antimicrobial Susceptibility Testing; Fifteenth Informational Supplement. Clinical and Laboratory Standards Institute
M11-A6 Vol. 24 No. 2 Methods for Antimicrobial Susceptibility Testing of Anaerobic Bacteria; Approved Standard-Sixth Edition. National Committee for Clinical Laboratory Standards.
M27-A2 Vol. 22 No. 15 Reference Method for Broth Dilution Antifungal Susceptibility Testing of Yeasts; Approved Standard-Second Edition. National Committee for Clinical Laboratory Standards.
M38-A Vol. 22 No. 16 Reference Method for Broth Dilution Antifungal Susceptibility Testing of Filamentous Fungi; Approved Standard. National Committee for Clinical Laboratory Standards.

MIC estimations were performed using wet plates, containing the LTX-109, prepared at GR Micro Ltd.

Cation-adjusted Mueller-Hinton broth (Oxoid Ltd., Basingstoke, UK and Trek Diagnostic Systems Ltd., East Grinstead, UK) (supplemented with 5% laked horse blood for Streptococcus spp.) was used for aerobic bacteria, with an initial inoculum of approximately 10⁵ colony-forming units (CFU)/mL.

Haemophilus test medium (Mueller-Hinton broth containing 0.5% yeast extract and Haemophilus test medium supplement which contains 15 mg/L of each of haematin and NAD, all obtained from Oxoid Ltd., Basingstoke, UK) was used for the Haemophilus influenzae and inoculated with approximately 10⁵ CFU/mL.

Supplemented Brucella broth (SBB) was used for the anaerobic strains with an inoculum of approximately 10⁶ CFU/mL. SBB is a broth consisting of 1% peptone, 0.5% ‘Lab-lemco’, 1% glucose and 0.5% sodium chloride supplemented with 5 μg/L haemin and 1 μg/L vitamin K (both obtained from Sigma Aldrich Ltd.)

Yeast and filamentous fungal MIC were performed in MOPS buffered RPMI 1640 medium (MOPS buffer obtained from Sigma Aldrich Ltd., RPMI 1640 obtained from Invitrogen Ltd, Paisley, Scotland). The yeast inocula were in the range 7.5×10²-4×10³ CFU/mL and the filamentous fungi approximately 8×10³-1×10⁵ CFU/mL.

Following normal practice all the plates containing Mueller-Hinton broth were prepared in advance, frozen at −70° C. on the day of preparation and defrosted on the day of use. Fungal, Haemophilus and anaerobic MIC determinations were all performed in plates prepared on the same day.

To evaluate whether freezing affected the activity of the LTX-109 some MIC determinations were repeated using plates containing freshly-prepared Mueller-Hinton broth.

Results

The results are shown below in Table 4 as a single line listing.

TABLE 4

| | LTX-109 |
|---|---|
| Species and Properties | MIC mg/L |
| Haemophilus influenzae - β-lactamase | 8 |
| negative clinical isolate | |
| Haemophilus influenzae - β-lactamase | 8 |
| positive clinical isolate | |
| Haemophilus influenzae - β-lactamase | 8 |
| negative, ampicillin resistant | |
| Streptococcus pneumoniae - penicillin | 32 |
| susceptible clinical isolate | |
| Streptococcus pneumoniae - penicillin | 32 |
| intermediate clinical isolate | |
| Streptococcus pneumoniae - penicillin | 16 |
| resistant clinical isolate | |
| Streptococcus pneumoniae - multi-drug | 32 |
| resistant clinical isolate | |
| Streptococcus pyogenes - macrolide (MLS) | 16 |
| resistant clinical isolate | |
| Moraxella catarrhalis - β-lactamase | 4 |
| positive clinical isolate | |
| Moraxella catarrhalis - reduced fluoroquinolone | 8 |
| susceptibility clinical isolate | |
| Pseudomonas aeruginosa - ATCC 27853 - | 8 |
| antibiotic-susceptible type strain | |
| Pseudomonas aeruginosa - ATCC 27853 - | 8 |
| antibiotic-susceptible type strain | |
| Pseudomonas aeruginosa - ATCC 27853 - | 8 |
| antibiotic-susceptible type strain | |
| Candida albicans - ATCC90028 - reference | 8 |
| strain | |
| Candida albicans - ATCC24433 - reference | 8 |
| strain | |
| Candida tropicalis - ATCC750 - reference | 4 |
| strain | |
| Candida parapsilosis - ATCC90018 - reference | 16 |
| strain | |
| Candida krusei - ATCC6258 - reference strain | 4 |
| Aspergillus niger - G. R. Micro collection | 8 |

For the triplicated experiments with Pseudomonas aeruginosa (ATCC 27853) two of the replicates were performed with frozen MHB plates and the other one with a fresh MHB plate. The data is highly consistent between replicates; freezing plates had no effect on the MIC observed. Freezing plates also had no effect on the MIC for other bacterial strains (data not shown).

The MIC data above indicates that LTX-109 has good antibacterial activity and antifungal activity against these clinically relevant bacteria and fungi.

### Example 5: Further Antibacterial Activity of LTX-109

This Example describes antibacterial activity of the compound LTX-109 against two library strains of Mycoplasma pneumoniae, a bacteria that can cause pneumonia.

Materials and Methods and Results

The compound LTX-109 has the structural formula as set out elsewhere herein.

Two library strains of Mycoplasma pneumoniae were used. These strains were stored at GR Micro Ltd. and maintained, with minimal sub-culture, deep frozen at −70° C. as a dense suspension in a high protein matrix of undiluted horse serum.

MIC determination was carried out using a broth microdilution method in Mycoplasma Broth Base (Oxoid Ltd, Hampshire, UK), prepared according to the manufacturer's instructions.

A MIC of 32 mg/L LTX-109 was determined for one of the library strains of Mycoplasma pneumoniae tested. A MIC of 16 mg/L LTX-109 was determined for the other of the library strains of Mycoplasma pneumoniae.

These data show that LTX-109 is active against Mycoplasma pneumoniae.

### Example 6: Antiviral Activity of 1% LTX-109 Against Rhinovirus

Aim

The aim of this study was to test the antiviral activity of 1% LTX-109 against Rhinovirus.

Methods

The Rhinovirus used was from BEI Resources: Rhinovirus 60, 2268-CV37 (Catalogue No. NR-51447).

To test whether 1% LTX-109 (w/v) has antiviral activity against Rhinovirus, 2.24×10⁵ infectious units of Rhinovirus (40 μl) were incubated with four volumes of 1% LTX-109 dissolved in PBS (160 μl), alongside a PBS (Phosphate-buffered saline) control and a 0.25% SDS (Sodium dodecyl sulphate) positive control. The treatments were performed in triplicate.

After 1 hour, the incubation was stopped by adding an excess of media, and the formulation was physically separated from the virus through a filter to reduce cytotoxicity on the assay cells. Some cloudiness in the preparation was observed. Infectious virus was quantified through a serial dilution (a series of ten-fold dilutions) on monolayers of HeLa cells in microtitre plates (HeLa cells are human cells capable of displaying a cytopathic effect (CPE) upon viral infection). For each dilution of the virus in the dilution series, eight wells of the microtitre plate were tested (i.e. each dilution was applied to eight separate wells, each well containing a HeLa cell monolayer). Appropriate controls were also performed. Seven days after infection of the cells, virus titre was quantified by determining the dilution at which half of the cells (half of the wells at a given dilution) displayed virus-induced cytopathic effect (TCID50). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to/ml of the starting solution (i.e. neat/undiluted solution) mentioned above.

A parallel test where the same procedure was carried out in the absence of virus was included to determine any residual cytotoxic effect of the formulation on the assay cells.

Results

The results of the test for antiviral activity of 1% LTX-109 against Rhinovirus are summarized in Table 5 (below).

After incubation with the PBS control for 1 hour, an average of 4.3 E+05 TCID50/ml of Rhinovirus was measured.

After 1 hour incubation with 1% LTX-109, an average of 4.8 E+03 TCID50/ml was measured, which corresponds to a decrease in infectivity of 1.9 logs, as compared to the PBS control.

After filtration, cytotoxicity on the HeLa cells was observed (only) with the neat application of the LTX-109 formulation (without virus), but without affecting the validity of the test.

TABLE 5
Average virus titres recovered after incubation
with PBS, 1% LTX-109 or 0.25% SDS for 1 hour.

| | | | | Log | |
|---|---|---|---|---|---|
| | Cytotoxicity | TCID50/ml ± SEM | Log | decrease | Percentage |
| PBS | No cytotoxicity | 4.34E+05 ± 1.28E+05 | 5.6 | | |
| 1% LTX- | Neat | 4.80E+03 ± 8.21E+02 | 3.7 | 1.9 | 98.89 |
| 109 | | | | | |
| SDS | Neat | 1.00E+02 | 2.0 | 3.6 | 99.98 |

### Conclusion

Based on the findings reported here, exposure of Rhinovirus to 1% LTX-109 for 1 hour in vitro caused a 1.9 log decrease in virus infectivity as compared to the PBS control, which corresponds to a ˜99% reduction as compared to the PBS control. These results show that LTX-109 has excellent antiviral activity against Rhinovirus (a non-enveloped virus).

SDS as a positive control provides a benchmark and confirms the suitability of the assay. Non-enveloped viruses are known to be susceptible to SDS and while the impact of SDS slightly exceeds that of LTX-109, the test peptide still performs well in comparison.

The cytotoxicity test shows that direct application of LTX-109 to the Hela cells is only cytotoxic before any serial dilutions are performed (i.e. with the neat formulation). Thus any residual peptide which may be associated with the virus after the filtration step is not responsible for the activity seen in the TCID50 assay.

### Example 7: Antiviral Activity of 3% LTX-109 Against Rhinovirus

Aim

The aim of this study was to test the antiviral activity of 3% LTX-109 against Rhinovirus.

Methods

The Rhinovirus used was from BEI Resources: Rhinovirus 60, 2268-CV37 (Catalogue No. NR-51447).

To test whether 3% LTX-109 (w/v) has antiviral activity against Rhinovirus, 9×10⁵ infectious units of Rhinovirus (40 μl) were incubated with four volumes of 3% LTX-109 dissolved in PBS (160 μl), alongside a PBS (Phosphate-buffered saline) control and a 0.25% SDS (Sodium dodecyl sulphate) positive control. The treatments were performed in triplicate.

After 1 hour, the incubation was stopped by adding 50 μl of mixture to 5 ml of media. Infectious virus was quantified through a serial dilution (a series of ten-fold dilutions) on a monolayer of HeLa cells in microtitre plates (HeLa cells are human cells capable of displaying a cytopathic effect (CPE) upon viral infection). For each dilution of the virus in the dilution series, eight wells of the microtitre plate were tested (i.e. each dilution was applied to eight separate wells, each well containing a HeLa cell monolayer). Appropriate controls were also performed. Seven days after infection of the cells, virus titre was quantified by determining the dilution at which half of the cells (half of the cells at a given dilution) displayed virus-induced cytopathic effect (TCID50). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to/ml of the starting solution (i.e. neat/undiluted solution) mentioned above.

A parallel test where the same procedure was carried out in the absence of virus was included to determine any residual cytotoxic effect of the formulation on the assay cells.

Results

The results of the test for antiviral activity of 3% LTX-109 against Rhinovirus are summarized in Table 6 (below).

After incubation with the PBS control for 1 hour, an average of 1.76×10⁴ TCID50/ml of Rhinovirus was measured.

After 1 hour incubation with 3% LTX-109, an average of 1.99×10² TCID50/ml was measured, which corresponds to a decrease in infectivity of 1.9 logs, as compared to the PBS control.

After dilution, cytotoxicity on the Hela cells was observed (only) with the neat application of the diluted 3% formulation (without virus), but without affecting the validity of the test.

TABLE 6
Average virus titres recovered after incubation
with PBS, 3% LTX-109 or 0.25% SDS for 1 hour.

| | | | | Log | |
|---|---|---|---|---|---|
| | Cytotoxicity | TCID50/ml ± SEM | Log | decrease | Percentage |
| PBS | No cytotoxicity | 1.76E+04 ± 5.63E+03 | 4.2 | | |
| 3% LTX-109 | Neat | 1.99E+02 ± 4.11E+01 | 2.3 | 1.9 | 98.87 |
| SDS | Neat | 1.58E+01 | 1.2 | 3.0 | 99.91 |

### Conclusion

Based on the findings reported here, exposure of Rhinovirus to 3% LTX-109 for 1 hour in vitro caused a 1.9 log decrease in virus infectivity as compared to the PBS control, which corresponds to a ˜99% reduction as compared to the PBS control. These results show that LTX-109 has excellent antiviral activity against Rhinovirus (a non-enveloped virus).

SDS as a positive control provides a benchmark and confirms the suitability of the assay. Non-enveloped viruses are known to be susceptible to SDS and while the impact of SDS slightly exceeds that of LTX-109, the test peptide still performs well in comparison.

The cytotoxicity test shows that direct application of LTX-109 to the Hela cells is only cytotoxic before any serial dilutions are performed (i.e. with the neat formulation). Thus any residual peptide which may be associated with the virus is not responsible for the activity seen in the TCID50 assay.

### Example 8: Antiviral Activity of LTX-12 Against the SARS-CoV-2 Virus

Aim

The aim of this study was to test the antiviral activity of LTX-12 against the SARS-CoV-2 virus.

Methods

The SARS-CoV-2 isolate used was from BEI Resources: SARS-CoV-2 isolate England/02/2020 (BEI Resources Catalogue Number (NR52359).

To test whether LTX-12 has antiviral activity against SARS-CoV-2, 7×10⁵ infectious units of SARS-CoV-2 (40 μl) were incubated with four volumes of 1% LTX-12 (w/v) dissolved in PBS (160 μl) or a PBS (Phosphate-buffered saline) negative control. As a positive control a buffer containing 0.2% Triton in PBS was tested in parallel. Each sample and the PBS control were tested in triplicates.

After 1 hour at room temperature (RT), the incubation was stopped by adding an excess of cold assay media (5 ml), and the formulation was physically separated from the virus through a filter (Sartorius VivaSpin 6, 100000 MWCO, PES (Sartorius, VS0642)) to reduce cytotoxicity on the assay cells. The assay media was M199 medium (Gibco, 41150087) supplemented with 0.4% BSA (Gibco 15260037) and 1×p/s (Gibco 15070063).

Infectious virus was quantified through a serial dilution (a series of ten-fold dilutions, 10⁻¹ to 10⁻⁸) on a monolayer of Vero cells plated in microtitre plates the day before at 8000 cells/100 μl/well (Vero cells are mammalian cells (African green monkey epithelial cells) capable of displaying a cytopathic effect (CPE) upon viral infection). The starting solution to make the serial dilution (i.e. the neat (or undiluted) solution) was obtained by re-suspending the virus that was separated via the filtration step in 1 ml of assay media, and then making serial dilutions of the starting solution (10⁻¹ to 10⁻⁸). For each dilution of the virus in the dilution series (10⁻¹ to 10⁻⁸), eight wells of the microtitre plate were tested (i.e. each dilution of virus was applied to eight separate wells, each well containing a Vero cell monolayer). Appropriate controls were also performed. Four days after infection of the cells, virus titre was quantified by determining the dilution at which half of the cells (half of the wells at a given dilution) displayed virus-induced cytopathic effect (TCID50), using the Reed and Muench method (L. J. Reed and H. Muench, American Journal of Epidemiology, Volume 27, Issue 3, 1938, Pages 493-497). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to/ml of the starting solution (i.e. neat/undiluted solution) mentioned above.

A parallel test where the same procedure was carried out in the absence of virus was included to determine any residual cytotoxic effect of LTX-12 on the assay cells.

Results

The results of the test for antiviral activity of LTX-12 against the SARS-CoV-2 virus are summarized in Table 7 (below).

After incubation with the PBS control for 1 h an average of 6.86 E+05 TCID50/ml of SARS-CoV-2 was measured.

After 1 h incubation with LTX-12, an average of 1.58 E+02 TCID50/ml was measured, corresponding to a decrease in infectivity of over 3 logs, or 99.977%, as compared to the PBS control.

After 1 h incubation with the Triton-based lysis buffer (positive control), 2.80 E+01 TCID50/ml was measured, corresponding to a decrease in infectivity of over 4 logs, or 99.996%, as compared to the PBS control.

In the above-mentioned parallel test, no significant cytotoxicity on the Vero cells (assay cells) was observed.

TABLE 7
Average virus titres recovered after incubation with PBS
or LTX-12. Virus titre recovered after incubation with Triton-
based lysis buffer (positive control) is also shown.

| CONTACT | | | Log | % |
|---|---|---|---|---|
| TIME: 1 h | Cytotoxicity | TCID50/ml ± SEM | decrease | Reduction |
| PBS | No cytotoxicity | 6.86E+05 ± 2.03E+05 | | |
| LTX-12 | No cytotoxicity | 1.58E+02 ± 0.00E+00 | 3.58 | 99.977 |
| POSITIVE | No cytotoxicity | 2.80E+01 | 4.34 | 99.996 |
| CONTROL | | (SEM ND) | | |

### Conclusions

Based on the findings reported here, exposure of SARS-CoV-2 to LTX-12 for 1 hour in vitro caused a decrease of about 3.6 logs in SARS-CoV-2 infectivity as compared to the PBS control, which corresponds to at least 99.9% reduction. These results show that LTX-12 has excellent antiviral activity against SARS-CoV-2. SARS-CoV-2 is a causative agent of pneumonia.

Triton as a positive control provides a benchmark and confirms the suitability of the assay. Enveloped viruses (such as SARS-CoV-2) are known to be susceptible to Triton and while the impact of Triton (positive control) slightly exceeds that of LTX-12, the test peptide (LTX-12) still performs well in comparison.

The cytotoxicity test shows that any residual peptide which may be associated with the virus after the filtration step is not responsible for the activity seen in the TCID50 assay.

### Example 9: Antiviral Activity of LTX-7 Against Influenza a Virus

Aim

The aim of this study was to test the antiviral activity of LTX-7 against Influenza A.

Methods

The Influenza A strain used was strain A/WSN/33 (H1N1).

To test whether LTX-7 has antiviral activity against Influenza A, 5×10⁵ infectious units of Influenza A (40 μl) were incubated with four volumes of 1% LTX-7 (w/v) dissolved in PBS or a PBS (Phosphate-buffered saline) negative control. As a positive control, a buffer containing 0.2% Triton X-100 in PBS was tested in parallel. Each sample and the PBS control were tested in triplicates.

After 1 hour at room temperature (RT), the incubation was stopped by adding an excess of cold assay media (5 ml), and the formulation was physically separated from the virus through a filter (Sartorius VivaSpin 6, 100,000 MWCO, PES (Sartorius, VS0642)) to reduce cytotoxicity on the assay cells. The assay media was DMEM (Gibco 61965-026) supplemented with 0.1% FBS (Gibco 10500-064), 20 mM Hepes (Gibco 15630-056), 0.3% BSA Fraction V (Gibco 15260037) and 1×p/s (Gibco 15070063).

Infectious virus was quantified through a serial dilution (a series of ten-fold dilutions, 10° to 10⁻⁷) on a monolayer of MDCK-II cells plated in microtitre plates the day before at 9,000 cells/100 μl/well (MDCK-II cells are mammalian cells (Madin-Darby canine kidney cells) capable of displaying a cytopathic effect (CPE) upon viral infection). The starting solution for the serial dilution (i.e. the neat (or undiluted) solution or 10° solution) was obtained by re-suspending the virus that was separated via the filtration step in 1 ml of assay media. For each dilution of the virus in the dilution series (10° to 10⁻⁷), eight wells of the microtitre plate were tested (i.e. each dilution of virus was applied to eight separate wells, each well containing a MDCK-II cell monolayer). Appropriate controls were also performed. Four days after infection of the cells, virus titre was quantified by determining the dilution at which half of the cells (half of the wells at a given dilution) displayed virus-induced cytopathic effect (TCID50), using the Reed and Muench method (L. J. Reed and H. Muench, American Journal of Epidemiology, Volume 27, Issue 3, 1938, Pages 493-497). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to/ml of the starting solution (i.e. neat/undiluted solution) mentioned above.

A parallel test where the same procedure was carried out in the absence of virus was included to determine any residual cytotoxic effect of LTX-7 on the assay cells.

Results

The results of the test for antiviral activity of LTX-7 against Influenza A virus (IAV) are summarized in Table 8 (below).

After incubation with the PBS control for 1 h an average of 1.33 E+06 TCID50/ml of Influenza A was measured.

After 1 h incubation with LTX-7, an average of 1.58 E+01 TCID50/ml was measured, which corresponds to a decrease in infectivity of over 4 logs, or at least 99.99%, as compared to the PBS control.

After 1 h incubation with the Triton X100 lysis buffer (positive control), 1.58 E+01 TCID50/ml was measured, corresponding to a decrease in infectivity of over 4 logs, or at least 99.99%, as compared to the PBS control.

After filtration, cytotoxicity on the MDCK-II cells was observed (only) with the neat application of the LTX-7 formulation, Triton X-100 (positive control) and PBS (without virus), but without affecting the validity of the test.

TABLE 8
Average virus titres recovered after incubation with PBS
or LTX-7. Virus titre recovered after incubation with Triton
X-100 lysis buffer (positive control) is also shown.

| CONTACT | | | Log | % | Rounded |
|---|---|---|---|---|---|
| TIME: 1 h | Cytotoxicity | TCID50/ml ± SEM | decrease | Reduction | % |
| PBS | neat | 1.33E+06 ± 7.64E+05 | | | |
| LTX-7 | neat | 1.58E+01 ± 0.00E+00 | 4.75 | 99.9988 | 99.99% |
| POSITIVE | neat | 1.58E+01 | 4.75 | 99.9988 | 99.99% |
| CONTROL | | (SEM ND) | | | |

### Conclusions

Based on the findings reported here, exposure of Influenza A to LTX-7 for 1 hour in vitro caused a decrease of 4.75-logs in Influenza A infectivity, as compared to the PBS control. This corresponds to at least 99.99% reduction. These results show that LTX-7 has excellent antiviral activity against Influenza A. Influenza A is a causative agent of sinusitis, pneumonia and otitis media.

Triton X-100 as a positive control provides a benchmark and confirms the suitability of the assay. Enveloped viruses (such as Influenza A) are known to be susceptible to Triton X-100. LTX-7 performs as well as the positive control in this study.

The cytotoxicity test shows that direct application of LTX-7 to the MDCK-II cells is only cytotoxic before any serial dilutions are performed (i.e. with the neat formulation). Thus any residual peptide which may be associated with the virus after the filtration step is not responsible for the activity seen in the TCID50 assay.

### Example 10: Antiviral Activity of LTX-7 Against Rhinovirus

Aim

The aim of this study was to test the antiviral activity of LTX-7 against Rhinovirus.

Methods

The Rhinovirus used was from BEI Resources: Rhinovirus (HRV-A60), Strain: 2268-CV37 (BEI Resources Catalogue Number NR-51447).

To test whether LTX-7 has antiviral activity against Rhinovirus, 5×10⁵ infectious units of Rhinovirus (40 μl) were incubated with four volumes of 1% LTX-7 (w/v) dissolved in PBS (160 μl) or a PBS (Phosphate-buffered saline) negative control. As a positive control, a buffer containing 0.25% SDS (Sodium dodecyl sulphate) in PBS was tested in parallel. Each sample and the PBS control were tested in triplicates.

After 1 hour at room temperature (RT), the incubation was stopped by adding an excess of cold assay media (5 ml), and the formulation was physically separated from the virus through a filter (Sartorius VivaSpin 6, 100,000 MWCO, PES (Sartorius, VS0642)) to reduce cytotoxicity on the assay cells. The assay media was DMEM (Gibco 61965-026) supplemented with 2% FBS (Gibco 10500-064), 20 mM Hepes (Gibco 15630-056) and 1×p/s (Gibco 15070063).

Infectious virus was quantified through a serial dilution (a series of ten-fold dilutions, 10° to 10⁻⁷) on a monolayer of HeLaM cells plated in microtitre plates the day before at ˜7,000 cells/100 μl/well (HeLaM cells are human cells capable of displaying a cytopathic effect (CPE) upon viral infection). The starting solution for the serial dilution (i.e. the neat (or undiluted) solution or 10° solution) was obtained by re-suspending the virus that was separated via the filtration step in 1 ml of assay media. For each dilution of the virus in the dilution series (10° to 10⁻⁷), eight wells of the microtitre plate were tested (i.e. each dilution was applied to eight separate wells, each well containing a HeLaM cell monolayer). Appropriate controls were also performed. Seven days after infection of the cells, virus titre was quantified by determining the dilution at which half of the cells (half of the cells at a given dilution) displayed virus-induced cytopathic effect (TCID50), using the Reed and Muench method (L. J. Reed and H. Muench, American Journal of Epidemiology, Volume 27, Issue 3, 1938, Pages 493-497). The TCID50 (TCID50/ml) assay (Tissue Culture Infectious Dose 50 assay) is a type of endpoint dilution assay that is well known in the art and routinely used to quantitatively measure virus titres. TCID50/ml provides a measure of infectious units of virus/ml. “/ml” refers to/ml of the starting solution (i.e. neat/undiluted solution) mentioned above.

A parallel test where the same procedure was carried out in the absence of virus was included to determine any residual cytotoxic effect of LTX-7 on the assay cells.

Results

The results of the test for antiviral activity of LTX-7 against Rhinovirus are summarized in Table 9 (below).

After incubation with the PBS control for 1 hour, an average of 9.17 E+04 TCID50/ml of Rhinovirus was measured.

After 1 hour incubation with LTX-7, an average of 3.86 E+03 TCID50/ml was measured, which corresponds to a decrease in infectivity of 1.33 logs, or at least 90%, as compared to the PBS control.

After 1 hour incubation with SDS (positive control), an average of 1.58 E+01 TCID50/ml was measured, which corresponds to a decrease in infectivity of 3.67 logs, or ˜99.9%, as compared to the PBS control.

After filtration, cytotoxicity on the HeLaM cells was observed (only) with the neat application of the LTX-7 formulation and SDS (without virus), but without affecting the validity of the test.

TABLE 9
Average virus titres recovered after incubation with PBS or LTX-7.
Virus titre recovered after incubation with SDS is also shown.

| CONTACT | | | Log | % | Rounded |
|---|---|---|---|---|---|
| TIME: 1 h | Cytotoxicity | TCID50/ml ± SEM | decrease | Reduction | % |
| PBS | No cytotoxicity | 9.17E+04 ± 3.76E+04 | | | |
| LTX-7 | Neat | 3.86E+03 ± 1.14E+03 | 1.33 | 95.791 | 90% |
| POSITIVE | Neat | 1.58E+01 | 3.67 | 99.983 | 99.9% |
| CONTROL | | (SEM ND) | | | |
| (SDS) | | | | | |

### Conclusions

Based on the findings reported here, exposure of Rhinovirus to LTX-7 for 1 hour in vitro caused a decrease of 1.33-logs in Rhinovirus infectivity, as compared to the PBS control. This corresponds to at least 90% reduction. These results show that LTX-7 has excellent antiviral activity against Rhinovirus. Rhinovirus can cause sinusitis, pneumonia and otitis.

SDS as a positive control provides a benchmark and confirms the suitability of the assay. Non-enveloped viruses (such as Rhinovirus) are known to be susceptible to SDS and while the impact of SDS exceeds that of LTX-7, the test peptide (LTX-7) still performs well in comparison.

The cytotoxicity test shows that direct application of LTX-7 to the HeLaM cells is only cytotoxic before any serial dilutions are performed (i.e. with the neat formulation). Thus any residual peptide which may be associated with the virus after the filtration step is not responsible for the activity seen in the TCID50 assay.

### Example 11: Antibacterial and Antifungal Activity of LTX-7

Example 4 herein describes antibacterial activity and antifungal activity of the compound LTX-109 against certain species of bacteria and fungi that can cause sinusitis, pneumonia and/or otitis.

Analogous experiments to those described in Example 4 were also performed with the compound LTX-7 (instead of LTX-109), and described below is antibacterial activity and antifungal activity of the compound LTX-7 against certain of those bacteria and fungi.

Results (Minimum Inhibitory Concentration, MIC) are shown below in Table 10 as a single line listing:

TABLE 10

| | LTX-7 |
|---|---|
| Species and Properties | MIC mg/L |
| Streptococcus pneumoniae - penicillin | 32 |
| intermediate clinical isolate | |
| Streptococcus pneumoniae - penicillin | 32 |
| resistant clinical isolate | |
| Streptococcus pyogenes - macrolide | 32 |
| (MLS) resistant clinical isolate | |
| Moraxella catarrhalis - β-lactamase | 4 |
| positive clinical isolate | |
| Moraxella catarrhalis - reduced | 8 |
| fluoroquinolone susceptibility clinical isolate | |
| Pseudomonas aeruginosa - ATCC 27853 - | 16 |
| antibiotic-susceptible type strain | |
| Pseudomonas aeruginosa - ATCC 27853 - | 32 |
| antibiotic-susceptible type strain | |
| Pseudomonas aeruginosa - ATCC 27853 - | 8 |
| antibiotic-susceptible type strain | |
| Candida albicans - ATCC90028 - | 2 |
| reference strain | |
| Candida albicans - ATCC24433 - | 2 |
| reference strain | |
| Candida tropicalis - ATCC750 - | 2 |
| reference strain | |
| Candida parapsilosis - ATCC90018 - | 4 |
| reference strain | |
| Candida krusei - ATCC6258 - | 4 |
| reference strain | |
| Aspergillus niger - G. R. Micro | 4 |
| collection | |

The MIC data above indicates that LTX-7 has good antibacterial activity and antifungal activity against these clinically relevant bacteria and fungi.

Example 5 herein describes antibacterial activity of the compound LTX-109 against two library strains of Mycoplasma pneumoniae, a bacteria that can cause pneumonia. Analogous experiments to those described in Example 5 were also performed with the compound LTX-7 (instead of LTX-109), and described below is antibacterial activity of LTX-7 against the two library strains of Mycoplasma pneumoniae

A MIC of 32 mg/L LTX-7 was determined for both of the library strains of Mycoplasma pneumoniae tested. These data show that LTX-7 is active against Mycoplasma pneumoniae.

1. A compound for use in the treatment of sinusitis, pneumonia or otitis in a subject, wherein said compound is a compound of Formula (I)
AA-AA-AA-X—Y—Z (I)
wherein, in any order, 2 of said AA (amino acid) moieties are cationic amino acids and 1 of said AA is an amino acid with a lipophilic R group, the R group having 14-27 non-hydrogen atoms;
X is a N atom, which may be substituted by a branched or unbranched C₁-C₁₀ alkyl or aryl group which group may incorporate up to 2 heteroatoms selected from N, O and S;
Y represents a group selected from —Rₐ—R_{b}—, —Rₐ—R_{b}—R_{b}— and —R_{b}—R_{b}—Rₐ— wherein
Rₐ is C, O, S or N, and
R_{b} is C; each of Rₐ and R_{b} may be substituted by C₁-C₄ alkyl groups or unsubstituted; and
Z is a group comprising 1 to 3 cyclic groups each of 5 or 6 non-hydrogen atoms, 2 or more of the cyclic groups may be fused and one or more of the cyclic groups may be substituted; the Z moiety incorporates a maximum of 15 non-hydrogen atoms; and wherein
the bond between Y and Z is a covalent bond between Rₐ or R_{b} of Y and a non-hydrogen atom of one of the cyclic groups of Z.

2. The compound for use according to claim 1, wherein said compound is a peptide.

3. The compound for use according to claim 1 or claim 2, wherein said cationic amino acids are lysine and/or arginine.

4. The compound for use according to any one of claims 1 to 3, wherein said cationic amino acids are arginine.

5. The compound for use according to any one of claims 1 to 4, wherein the lipophilic R group contains 2 or more cyclic groups which may be fused or connected.

6. The compound for use according to any one of claims 1 to 5, wherein X is unsubstituted.

7. The compound for use according to any one of claims 1 to 6, wherein Rₐ is C.

8. The compound for use according to any one of claims 1 to 7, wherein Y is —Rₐ—R_{b}— and unsubstituted.

9. The compound for use according to any one of claims 1 to 8, wherein Y is —CH₂—CH₂—.

10. The compound for use according to any one of claims 1 to 9, wherein Z is phenyl.

11. The compound for use according to any one of claims 1 to 10, wherein said compound is a compound of formula (II)
AA₁-AA₂-AA₁-X—Y—Z (II)
wherein:
AA₁ is a cationic amino acid;
AA₂ is an amino acid with a lipophilic R group, the R group having 14-27 non-hydrogen atoms; and
X, Y and Z are as defined in any one of claims 1 to 10.

12. The compound for use according to any one of claims 1 to 11, wherein the amino acid with a lipophilic R group is selected from tributyl tryptophan (Tbt) or a biphenylalanine derivative selected from Phe (4-(2-Naphthyl)), Phe (4-(1-Naphthyl)), Bip (4-n-Bu), Bip (4-Ph) and Bip (4-T-Bu).

13. The compound for use according to any one of claims 1 to 12, wherein the amino acid with a lipophilic R group is tributyl tryptophan (Tbt).

14. The compound for use according to any one of claims 1 to 13, wherein —X—Y—Z together are —NHCH₂CH₂Ph.

15. The compound for use according to any one of claims 1 to 14, wherein said compound has the structural formula:

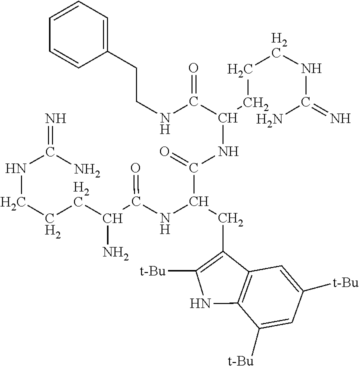 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

16. The compound for use according to any one of claims 1 to 15, wherein a causative agent of said sinusitis, pneumonia or otitis is a virus.

17. The compound for use according to any one of claims 1 to 16, wherein a causative agent of said sinusitis, pneumonia or otitis is a bacteria.

18. The compound for use according to any one of claims 1 to 17, wherein the type of infectious agent causing said sinusitis, pneumonia or otitis is not known prior to the first administration of said compound to said subject.

19. The compound for use according to any one of claims 1 to 18, wherein said subject is a human subject.

20. A pharmaceutical formulation comprising a compound as defined in any one of claims 1 to 15 and a diluent, carrier and/or excipient for use in the treatment of sinusitis, pneumonia or otitis in a subject.

21. The compound for use according to any one of claims 1 to 19 or the pharmaceutical composition for use according to claim 20, wherein said treatment is a therapeutic treatment.

22. The compound for use according to any one of claims 1 to 19 or the pharmaceutical composition for use according to claim 20, wherein said treatment is a prophylactic treatment.

23. A method of treating sinusitis, pneumonia or otitis in a subject, which method comprises administering to a subject in need thereof an effective amount of compound as defined in any one of claims 1 to 15.

24. The method of claim 23, wherein the type of infectious agent causing said sinusitis, pneumonia or otitis is not known prior to the first administration of said compound to said subject.

25. The method of claim 23 or claim 24, wherein said subject is a human subject.

26. Use of a compound as defined in any one of claims 1 to 15 in the manufacture of a medicament for use in the treatment of sinusitis, pneumonia or otitis.

27. The use of claim 26, wherein said treatment comprises administering to a subject in need thereof an effective amount of a compound as defined in any one of claims 1 to 15 and wherein the type of infectious agent causing said sinusitis, pneumonia or otitis is not known prior to the first administration of said compound to said subject.

28. The use of claim 26 or claim 27, wherein said subject is a human subject.

29. The compound for use according to any one of claims 1 to 19, 21 or 22, or the pharmaceutical composition for use according to any one of claims 20 to 22, or the method of any one of claims 23 to 25, or the use of any one of claims 26 to 28, wherein said otitis is otitis media or otitis externa.
